(19) United States
(12) Patent Application Publication
(10) Pub. No.: US 2015/0209009 A1
(43) Pub. Date: Jul. 30, 2015

(54) ULTRASOUND PROBE

(71) Applicant: Hitachi Metals, Ltd., Tokyo (JP)

(72) Inventors: Detian HUANG, Hitachi (JP); Takanobu WATANABE, Hitachi (JP); Norihiro NISHIURA, Hitachi (JP)

(21) Appl. No.: 14/308,282

(22) Filed: Jun. 18, 2014

(30) Foreign Application Priority Data
Jan. 24, 2014 (JP) 2014-011388

(51) Int. Cl. A61B 8/00; A61B 8/08

(52) U.S. Cl. CPC A61B 8/4444; A61B 8/4483; A61B 8/5207; A61B 8/5223

(57) ABSTRACT

An ultrasound probe includes a swingable ultrasound element, an electric circuit to be electrically connected with a probe cable, and a woven cable which electrically connects together the ultrasound element and the electric circuit.

The present application is based on Japanese patent application No.2014-011388 filed on Jan. 24, 2014, the entire contents of which are incorporated herein by reference.

### BACKGROUND OF THE INVENTION

1. Field of the Invention

This invention relates to an ultrasound probe suitable for acquiring a three dimensional ultrasound image.

2. Description of the Related Art

To diagnose a living body internally and noninvasively, an ultrasound diagnosis device is widely used, which images or visualizes internal conditions of the living body by transmitting an ultrasound wave internally from a surface of the living body and receiving the internally reflected wave from the living body.

As shown in FIG. 2, the ultrasound diagnosis device is equipped with an ultrasound probe 200 including an ultrasound element 201 comprising a plurality of piezoelectric elements (oscillators) arranged side by side (juxtaposed), an electric circuit 203 to be electrically connected with a probe cable 202, and an internal wiring 204 to electrically connect together the ultrasound element 201 and the electric circuit 203.

In order to acquire a three dimensional ultrasound image, the ultrasound element 201 is designed to, as shown in FIG. 3, swing at a rate of the order of 120 times/m in a direction perpendicular to a juxtaposing direction (width direction) of the plurality of piezoelectric elements, and scan an ultrasound transmitting location or receiving location.

On the other hand, the electric circuit 203 is fixed at a predetermined location in the ultrasound probe 200, and therefore the swinging of the ultrasound element 201 causes load to act on the internal wiring 204 connecting between the ultrasound element 201 and the electric circuit 203.

As the internal wiring 204 capable of withstanding the load due to the swinging of the ultrasound element 201, the following examples are known: For example, a flat cable comprising a plurality of coaxial wires arranged side by side; a flexible printed circuit board produced by transferring a circuit pattern to a surface of an electrically insulating film.

### SUMMARY OF THE INVENTION

Because as with other electronic devices, the ultrasound probe 200 follows a course of miniaturization, a wiring length of the internal wiring 204 is often limited to 80 mm or shorter. Also, a swing angle of the ultrasound element 201 has reached 60 degrees so as to widen a scanning range of the ultrasound element 201.

In view of these circumstances, the internal wiring 204 is required to withstand the load due to the swinging of the ultrasound element 201 under more severe conditions than conventional conditions.

Under such conditions, when the flat cable is adopted as the internal wiring 204, the load due to the swinging of the ultrasound element 201 all acts on the coaxial wire having a maximally thinned diameter for ensuring the miniaturization of the ultrasound probe 200. The coaxial wire therefore tends to break.

Also, under similar conditions, when the flexible printed circuit board is adopted as the internal wiring 204, the load due to the swinging of the ultrasound element 201 is likely to form cracks in the electrically insulating film, and the conductor is likely to break with the progress of the cracks. Also, when the flexible printed circuit board is electromagnetically shielded with an electrically conductive cloth or a shield tape therearound, its flexibility is likely to be spoiled and the smooth swinging of the ultrasound element 201 is likely to be prevented. There is therefore a limit on the enhancement of electrical characteristics.

Accordingly, it is an object of the present invention to provide an ultrasound probe, which is capable of satisfying a demand for miniaturization in recent years, widening a scanning range of an ultrasound element, and enhancing electrical characteristics.

(1) According to an embodiment of the invention, an ultrasound probe comprises:

a swingable ultrasound element;

an electric circuit to be electrically connected with a probe cable; and

a woven cable which electrically connects together the ultrasound element and the electric circuit.

In the embodiment, the following modifications and changes can be made.

(i) The woven cable is not more than 80 mm in wiring length.

(ii) The woven cable withstands 20 million or more swings when a swing angle of the ultrasound element is 60 degrees.

(iii) The woven cable stretches or compresses in a side by side arrangement direction of a plurality of electric wires thereof

(iv) The woven cable includes a plurality of electric wires arranged side by side, and a fiber member woven in such a manner as to sew between the plurality of the electric wires and in a side by side arrangement direction of the plurality of the electric wires, and the fiber member comprises a polyurethane elastic fiber.

(v) The fiber member is being elongated by weaving in such a manner as to sew between the plurality of the electric wires.

(vi) The fiber member comprises a monofilament.

(Points of the Invention)

The present invention can provide the ultrasound probe, which is capable of satisfying a demand for miniaturization in recent years, widening a scanning range of the ultrasound element, and enhancing electrical characteristics.

### BRIEF DESCRIPTION OF THE DRAWINGS

The preferred embodiments according to the invention will be explained below referring to the drawings, wherein:

FIG. 1 is a schematic plane cross section view showing an ultrasound probe in an embodiment according to the present invention;

FIG. 2 is a schematic plane cross section view showing a conventional ultrasound probe; and

FIG. 3 is a schematic cross-sectional view showing a conventional ultrasound probe.

### DETAILED DESCRIPTION OF THE EMBODIMENTS

Below is described an embodiment of the present invention according to an attached drawing.

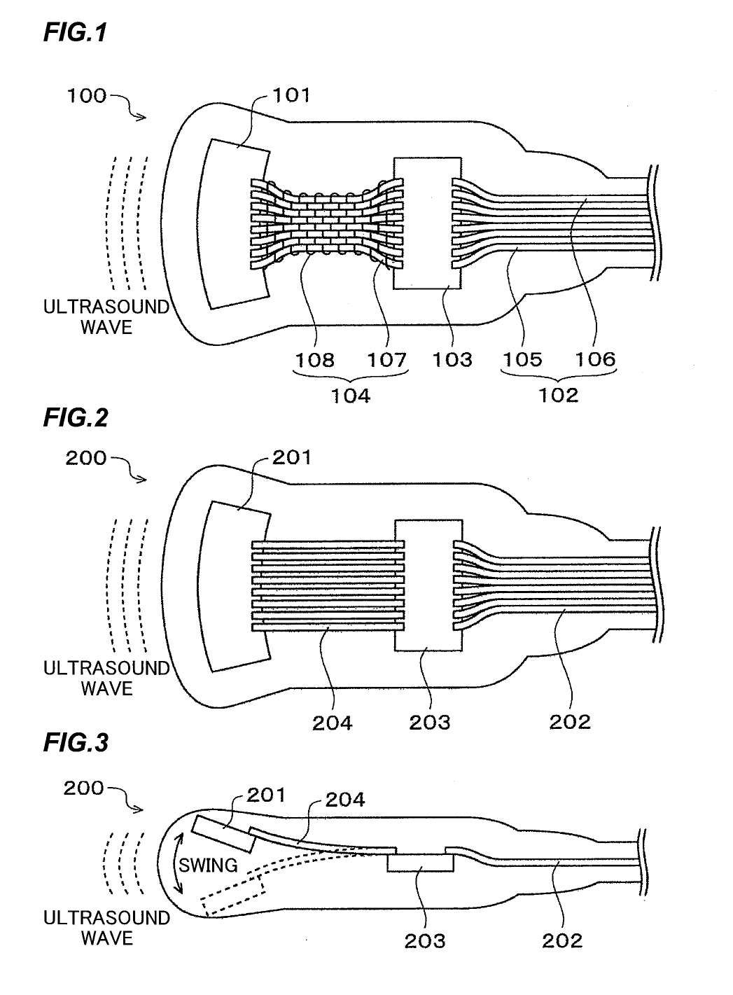

As shown in FIG. 1, an ultrasound probe 100 in the present embodiment is characterized by comprising a swingable ultrasound element 101, an electric circuit 103 to be electrically with a probe cable 102, and a woven cable 104 to electrically connect together the ultrasound element 101 and the electric circuit 103.

The ultrasound element 101 comprises a plurality of piezoelectric elements (oscillators) arranged side by side (juxtaposed), and in order to acquire a three dimensional ultrasound image, is designed to swing at a rate of the order of 120 times/m in a direction perpendicular to a juxtaposing direction of the plurality of piezoelectric elements, and scan an ultrasound transmitting location or receiving location. This ultrasound element 101 is mounted on a printed circuit board and the like.

The probe cable 102 comprises a plurality of signal wires 105 and a power wire 106 and electrically connects together a body portion of an ultrasound diagnosis device not shown and the ultrasound probe 100.

The electric circuit 103 comprises a printed circuit board constituting a drive circuit which drives the ultrasound element 101 and is fixed at a predetermined location inside the ultrasound probe 100.

The woven cable 104 has a plurality of electric wires 107 arranged side by side and a the fiber member 108 which is woven in such a manner as to sew between the plurality of electric wires 107 in a side by side arrangement direction of the plurality of the electric wires 107.

The electric wires 107 preferably comprises a coaxial wire with electromagnetic shielding applied thereto to enhance electrical characteristics, and these are arranged alternately to constitute the plurality of electric wires 107.

The coaxial wire includes an inner conductor made of a metal wire, an insulating layer formed around the inner conductor, an outer conductor formed of a metal wire laterally wrapped or braided around the insulating layer, and a jacket formed around the outer conductor.

The metal wire constituting the inner conductor and the outer conductor is formed of copper or aluminum having excellent electrical conductivity. Also, the metal wire may be a solid wire or a stranded wire, and its surface may be plated.

The insulating layer and the jacket are formed of a fluoric resin such as tetrafluoroethylene perfluoroalkyl vinyl ether copolymer (PFA), tetrafluoroethylene hexafluoropropylene copolymer (FEP) or ethylene tetrafluoroethylene copolymer (ETFE), or polyethylene terephthalate (PET).

The fiber member 108 zigzags between the plurality of electric wires 107, from one side to the other side in a width direction (side by side arrangement direction of the plurality of electric wires 107) and from one longitudinal end to the other longitudinal end of the woven cable 104, and is woven so as to constrain and fix the plurality of electric wires 107 in a flat shape.

At this point, the fiber member 108 is preferably woven in such a manner as to sew one electric wire 107 as one unit in the middle in the width direction of the woven cable 104.

It should be noted that the middle in the width direction of the woven cable 104 is not limited to on a central axis of the woven cable 104, but conceptually includes vicinity thereof.

With these configurations, all the electric wires 107 constituting the woven cable 104 are constrained to the fiber member 108, and the plurality of electric wires 107 are arranged in such a manner as to huddle together and are aligned at a uniform wiring pitch. Therefore the width of the woven cable 104 is small and is able to contribute to miniaturization of the ultrasound probe 100.

Also, the fiber member 108 is woven in the entire longitudinal direction of the woven cable 104, but in order to facilitate connection with the ultrasound element 101 or the electric circuit 103, the fiber member 108 woven at both longitudinal ends of the woven cable 104 may be removed.

At this point, without special work to dissolve the fiber member 108 with a solvent, the electric wires 107 and the fiber member 108 are easily separated only by pulling a tip of the fiber member 108. It is therefore possible to simplify connection with the ultrasound element 101 or the electric circuit 103, and reduce a burden on a user.

The fiber member 108 is made of a fiber having high elongation and low initial modulus, more specifically, a polyurethane elastic fiber having a high elongation of not less than 500% and not more than 900%, an elongation recovery rate at the time of 300% elongation of not less than 90%, and an initial modulus for 300% elongation of not less than 5 cN/dtex and not more than 30 cN/dtex.

The reason for setting the elongation at not less than 500% and not more than 900% is because at less than 500%, when the woven cable 104 is bent, or when the alignment pitch of the plurality of electric wires 107 is longitudinally partially changed depending on a desired layout, the fiber member 108 cannot sufficiently follow that bending or change.

Also, it is because at more than 900% the function of the fiber member 108 for constraining and fixing the plurality of electric wires 107 lowers.

The reason for setting the elongation recovery rate at the time of 300% elongation at not less than 90% is because at less than 90%, when the woven cable 104 is bent, the fiber member 108 is fully stretched, causing the woven cable 104 to be unlikely to return to its shape before its bending.

The reason for setting the initial modulus for 300% elongation at not less than 5 cN/dtex and not more than 30 cN/dtex is because at less than 5 cN/dtex, when the fiber member 108 is woven between the plurality of electric wires 107, the plurality of electric wires 107 cannot sufficiently be constrained with the fiber member 108, and the woven cable 104 in a good shape cannot be produced, but requires a separate subsequent step for arranging a good shape of the woven cable 104, leading to a rise in production cost.

Also, it is because at more than 30 cN/dtex, when the fiber member 108 is woven between the plurality of electric wires 107, the fiber member 108 strongly tightens the electric wires 107, being likely to cause undulation and break resulting therefrom in the electric wires 107, worsening electrical characteristics thereof.

That is, setting the initial modulus for 300% elongation at not less than 5 cN/dtex and not more than 30 cN/dtex allows the fiber member 108 to be woven between the plurality of electric wires 107 with no extra load acting on the electric wires 107.

As the polyurethane elastic fiber meeting these conditions, there is ROICA (trademark, Asahi Kasei Fibers Corporation), for example. Also, the fiber member 108 is made of preferably a monofilament from the point of view of textile strength enhancement and thinning of the woven cable 104.

The use of this polyurethane elastic fiber as the fiber member 108 allows the fiber member 108 to have very thin fineness, be substantially elongated and woven between the plurality of electric wires 107.

For example, it is possible to weave the fiber member 108 having not less than 17 dtex and not more than 45 dtex by elongation to 300%. The outer diameter of the 300% elongated fiber member 108 having not less than 17 dtex and not more than 45 dtex is 0.04 mm or less.

And after weaving the fiber member 108, the plurality of electric wires 107 are arranged in such a manner as to huddle together by elongation recovery of the fiber member 108, but because the elongation of the fiber member 108 is high, no excessive force is applied to the plurality of electric wires 107.

Therefore, even when the outer diameter of the electric wires 107 is small, the elongation recovery of the fiber member 108 allows the fiber member 108 to be woven between the plurality of electric wires 107 with no stress causing a small bend in the electric wires 107, and with no undulation and break resulting therefrom being caused in the electric wires 107.

This allows for shortening the separation (wiring pitch) between the adjacent electric wires 107 with no extra load acting on the electric wires 107, and thereby narrowing the width of the woven cable 104, compared with the conventional width.

Furthermore, the fiber member 108 formed of the previously described polyurethane elastic fiber is able to sufficiently elongate even after being woven between the plurality of electric wires 107. Therefore, the fiber member 108 allows the woven cable 104 to serve to stretch or compress in the width direction thereof.

This allows the woven cable 104 to have sufficient flexibility and follow the swinging of the ultrasound element 101 in the condition that the swing angle of the ultrasound element 101 reaches 60 degrees, even if the wiring length of the woven cable 104 is 80 mm or less.

Also, the fiber member 108 is woven preferably at a texture density of not less than 20 turns and not more than 30 turns per 1 cm in the cable longitudinal direction of the woven cable 104. The reason for weaving the fiber member 108 at a texture density of not less than 20 turns and not more than 30 turns per 1 cm in the cable longitudinal direction of the woven cable 104 is because if the texture density is less than 20 turns per 1 cm, the plurality of electric wires 107 cannot sufficiently be bundled together, while if the texture density exceeds 30 turns per 1 cm, the flexibility of the woven cable 104 is spoiled.

This woven cable 104 allows most of the load due to the swinging of the ultrasound element 101 to escape to the very elastic fiber member 108. Therefore, even if a coaxial wire having excellent electrical characteristics and a maximally thinned diameter is adopted, the woven cable 104 can withstand the load due to the swinging of the ultrasound element 101 under more severe conditions than conventional conditions, and thereby prevent the break of the coaxial wire, and also no problem in adopting a flexible printed circuit board arises.

Furthermore, in the woven cable 104, the plurality of electric wires 107 are made integral by the fiber member 108. Therefore, the plurality of electric wires 107 are unlikely to be separated, even if exposed to swinging of the ultrasound element 101 under more severe conditions than conventional conditions. This prevents the electric wires 107 from deviating from an original course, slipping, projecting from the woven cable 104 and being acted on by excessive load.

Therefore, no variation in the break life of the electric wire 107 is likely to occur. It is possible to substantially enhance the break life itself as compared with the conventional electric wire. Specifically, the electric wire 107 can withstand 20 million or more swings, when the wiring length of the woven cable 104 is 80 mm or less and the swing angle of the ultrasound element 101 is 60 degrees.

Note that connecting the woven cable 104 to a printed circuit board with the ultrasound element 101 mounted thereon and to both front and back sides of the electric circuit 103 allows packaging density to be doubly enhanced.

As described above, the present invention can provide the ultrasound probe 100 which is capable of satisfying a demand for miniaturization in recent years, widening a scanning range of the ultrasound element, and enhancing electrical characteristics.

Although the invention has been described with respect to the specific embodiments for complete and clear disclosure, the appended claims are not to be thus limited but are to be construed as embodying all modifications and alternative constructions that may occur to one skilled in the art which fairly fall within the basic teaching herein set forth.

What is claimed is:

1. An ultrasound probe, comprising:
a swingable ultrasound element;
an electric circuit to be electrically connected with a probe cable; and
a woven cable which electrically connects together the ultrasound element and the electric circuit.

2. The ultrasound probe according to claim 1, wherein the woven cable is not more than 80 mm in wiring length.

3. The ultrasound probe according to claim 1, wherein the woven cable withstands 20 million or more swings when a swing angle of the ultrasound element is 60 degrees.

4. The ultrasound probe according to claim 1, wherein the woven cable stretches or compresses in a side by side arrangement direction of a plurality of electric wires thereof.

5. The ultrasound probe according to claim 1, wherein the woven cable includes a plurality of electric wires arranged side by side, and a fiber member woven in such a manner as to sew between the plurality of the electric wires and in a side by side arrangement direction of the plurality of the electric wires, and the fiber member comprises a polyurethane elastic fiber.

6. The ultrasound probe according to claim 5, wherein the fiber member is being elongated by weaving in such a manner as to sew between the plurality of the electric wires.

7. The ultrasound probe according to claim 5, wherein the fiber member comprises a monofilament.
